(19) United States
(12) Patent Application Publication
(10) Pub. No.: US 2026/0029520 A1
(43) Pub. Date: Jan. 29, 2026

(54) ULTRASONIC DIAGNOSTIC APPARATUS, METHOD OF SIGNAL PROCESSING, AND NON-TRANSITORY COMPUTER READABLE MEDIUM

(71) Applicant: CANON MEDICAL SYSTEMS CORPORATION, Tochigi (JP)

(72) Inventor: Hiroki TAKAHASHI, Nasushiobara (JP)

(73) Assignee: CANON MEDICAL SYSTEMS CORPORATION, Tochigi (JP)

(21) Appl. No.: 19/278,961

(22) Filed: Jul. 24, 2025

(30) Foreign Application Priority Data
Jul. 24, 2024 (JP) 2024-118737

(51) Int. Cl. G01S 7/52; A61B 8/14

(52) U.S. Cl. CPC G01S 7/52028; A61B 8/14

(57) ABSTRACT

An ultrasonic diagnostic apparatus according to an embodiment includes a transmitter circuit and receiver circuit, and a processing circuit. The transmitter circuit and receiver circuit transmits an ultrasonic wave into a subject, based on a transmission condition, and receives an echo from inside the subject. The processing circuit causes the ultrasonic wave transmitted based on the transmission condition to propagate forward to obtain a transmission wave field, causes a signal based on the echo to propagate backward to obtain a reception wave field by applying a weight function depending on a wavefront incident angle, and performs correlation analysis between the transmission wave field and the reception wave field to generate an echo component.

### CROSS-REFERENCE TO RELATED APPLICATIONS

This application is based upon and claims the benefit of priority from Japanese Patent Application No. 2024-118737, filed on Jul. 24, 2024; the entire contents of which are incorporated herein by reference.

### FIELD

Embodiments described herein relate generally to an ultrasonic diagnostic apparatus, a method of signal processing, and a non-transitory computer readable medium.

### BACKGROUND

In the field of seismic wave measurement, seismic wave interferometry (also referred to as shot-profile migration (SPM) or wave-field correlation) or RTM (Reverse-Time Migration) is known. Seismic wave interferometry is a method for reconstructing a hypocentral position and reflection surfaces in the ground, and is a method for estimating the hypocentral position and the like by performing correlation processing between a transmission wave field obtained from forward propagation simulation and a reception wave field obtained from back propagation simulation.

Since a seismic wave and an ultrasonic wave are common in terms of being an elastic wave, the method of seismic wave interferometry may be used for image reconstruction in an ultrasonic diagnostic apparatus to reconstruct an ultrasonic image.

A reception sensor of the ultrasonic diagnostic apparatus generally includes a plurality of piezoelectric elements. Since the frequency used in the ultrasonic diagnostic apparatus is in the order of a few megahertz to a few tens of megahertz and the aperture width of the sensor is the order of a few centimeters, it can be said that tissue in a living body is positioned on a complex short-distance sound field. In such a condition, by performing image reconstruction while partially limiting the reflected wavefront from a point that wants to be imaged, it may be possible to suppress unnecessary artifacts caused by signals from spaces other than the point desired. When a case in which the depth of the point to be imaged is shallow is considered, the amplitude of an ultrasonic wave itself has sufficient intensity. Thus, for example, to prevent overlooking of a tumor or the like on the body surface, it is desirable to perform image reconstruction using only a component in a direction directed to the sensor out of a reflected wavefront from the tumor and to suppress components in other directions because they contain many unnecessary wave signals from outside the tumor. In contrast, when the point to be imaged is deep, the amplitude of the ultrasonic wave becomes smaller due to signal attenuation. Thus, to ensure the S/N ratio, image reconstruction is desirably performed using all the signals received by the sensor.

However, since conventional seismic wave interferometry performs image reconstruction using signals in various directions received by the sensor with equivalent weights, it may be difficult to ensure the S/N ratio while suppressing unnecessary wave components.

### BRIEF DESCRIPTION OF THE DRAWINGS

FIG. 1 is a diagram of an example of the configuration of an ultrasonic apparatus according to an embodiment;

FIG. 2 is a flowchart of an example of the flow of processing performed by an ultrasonic diagnostic apparatus according to a first embodiment;

FIG. 3 is a diagram of an example of the processing performed by the ultrasonic diagnostic apparatus according to the first embodiment;

FIG. 4 is a flowchart illustrating the processing at Step S300A in FIG. 2 in more detail;

FIG. 5 is a diagram illustrating the processing performed by the ultrasonic diagnostic apparatus according to the first embodiment;

FIG. 6 is a diagram illustrating the processing performed by the ultrasonic diagnostic apparatus according to the first embodiment;

FIG. 7 is a diagram illustrating the processing performed by the ultrasonic diagnostic apparatus according to the first embodiment;

FIG. 8 is a flowchart illustrating the flow of processing performed by an ultrasonic diagnostic apparatus according to a second embodiment;

FIG. 9 is a diagram illustrating the processing performed by the ultrasonic diagnostic apparatus according to the second embodiment;

FIG. 10 is a diagram illustrating the processing performed by the ultrasonic diagnostic apparatus according to the second embodiment; and

FIG. 11 is a flowchart illustrating the processing performed by the ultrasonic diagnostic apparatus according to the second embodiment.

### DETAILED DESCRIPTION

An ultrasonic diagnostic apparatus provided in an aspect of the present invention includes a transmitter circuit and receiver circuit, and a processing circuit. The transmitter circuit and receiver circuit transmits an ultrasonic wave into a subject, based on a transmission condition, and receives an echo from inside the subject. The processing circuit causes the ultrasonic wave transmitted based on the transmission condition to propagate forward to obtain a transmission wave field, causes a signal based on the echo to propagate backward to obtain a reception wave field by applying a weight function depending on a wavefront incident angle, and performs correlation analysis between the transmission wave field and the reception wave field to generate an echo component.

Embodiments of an ultrasonic diagnostic apparatus, a method of signal processing, and a computer program will be described below in detail with reference to the drawings.

### First Embodiment

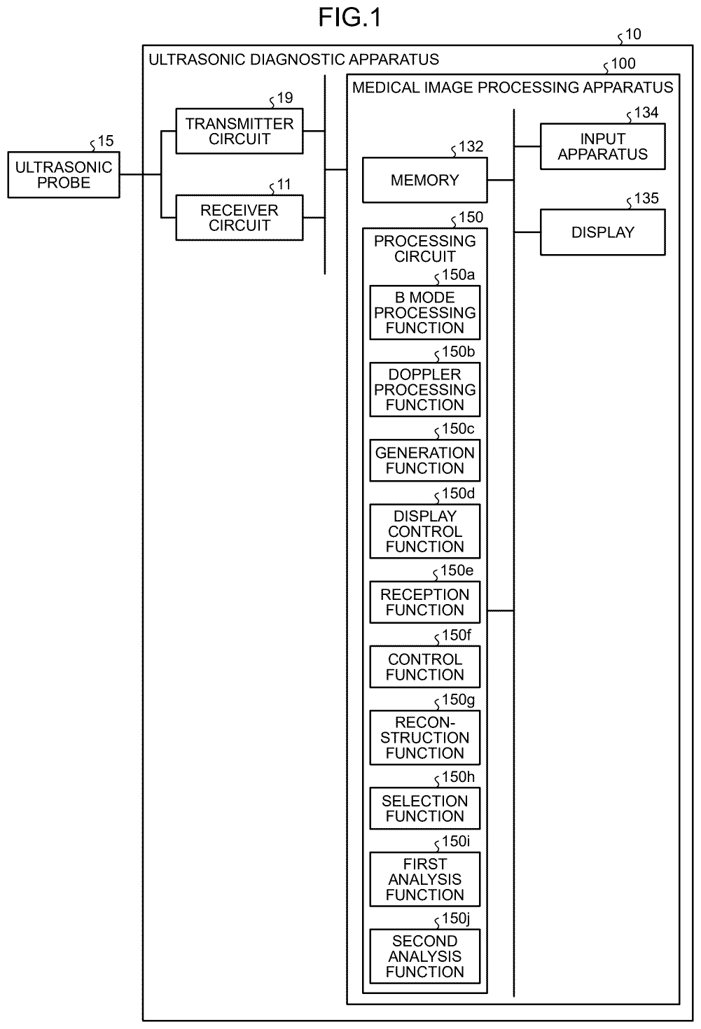

First, the configuration of an ultrasonic diagnostic apparatus according to a first embodiment will be described. FIG. 1 is a block diagram of a configuration example of the ultrasonic diagnostic apparatus according to the first embodiment. As exemplified in FIG. 1, the ultrasonic diagnostic apparatus according to the first embodiment includes an ultrasonic probe 15 and an ultrasonic diagnostic apparatus 10. The ultrasonic diagnostic apparatus 10 includes a transmitter circuit 19, a receiver circuit 11, and a medical image processing apparatus 100.

The ultrasonic probe 15 includes a plurality of piezoelectric transducer elements, and these piezoelectric transducer elements generate an ultrasonic wave based on a drive signal supplied from the transmitter circuit 19 of the ultrasonic diagnostic apparatus 10, which will be described later. The piezoelectric transducer elements of the ultrasonic probe 15 receive a reflected wave from a subject P, and convert it into an electric signal (a reflected wave signal). The ultrasonic probe 15 includes a matching layer provided in the piezoelectric transducer elements, a backing material preventing the propagation of the ultrasonic wave backward from the piezoelectric transducer elements, and the like. Note that the ultrasonic probe 15 is detachably connected to the ultrasonic diagnostic apparatus 10.

When the ultrasonic wave is transmitted from the ultrasonic probe 15 to the subject P, the transmitted ultrasonic wave is successively reflected on discontinuities in the acoustic impedance in the body tissue of the subject P, received as a reflected wave by the piezoelectric transducer elements of the ultrasonic probe 15, and converted into a reflected wave signal. The amplitude of the reflected wave signal depends on the difference in acoustic impedance at the discontinuity on which the ultrasonic wave is reflected. Note that when a transmitted ultrasonic pulse is reflected on a moving blood flow or a surface such as the heart wall, a frequency shift occurs in the reflected wave signal because of the Doppler effect depending on the velocity component of a moving body with respect to an ultrasonic transmission direction.

Note that the embodiment is applicable when the ultrasonic probe 15 is either a 1D array probe, which scans the subject P in two dimensions, or a mechanical 4D probe or a 2D array probe, which scans the subject P in three dimensions.

The ultrasonic diagnostic apparatus 10 is an apparatus generating ultrasonic image data based on the reflected wave signal received from the ultrasonic probe 15. The ultrasonic diagnostic apparatus 10 illustrated in FIG. 1 is an apparatus that can generate two-dimensional ultrasonic image data based on a two-dimensional reflected wave signal and can generate three-dimensional ultrasonic image data based on a three-dimensional reflected wave signal. However, the embodiment is applicable even when the ultrasonic diagnostic apparatus 10 is an apparatus dedicated for two-dimensional data.

As exemplified in FIG. 1, the ultrasonic diagnostic apparatus 10 includes the transmitter circuit 19, the receiver circuit 11, and the medical image processing apparatus 100.

The transmitter circuit 19 and the receiver circuit 11 control ultrasonic transmission and reception performed by the ultrasonic probe 15 based on instructions by a processing circuit 150 including a control function 150f, which will be described later. The transmitter circuit 19 includes a pulse generator, a transmission delay unit, a pulser, and the like, and supplies a drive signal to the ultrasonic probe 15. The pulse generator repeatedly generates rate pulses for forming a transmission ultrasonic wave at a certain pulse repetition frequency (PRF). A delay time for each piezoelectric transducer element is necessary to focus the ultrasonic wave generated from the ultrasonic probe 15 into a beam shape and to determine transmission directivity. The transmission delay unit gives the delay time to each rate pulse generated by the pulse generator. The pulser applies a drive signal (a drive pulse) to the ultrasonic probe 15 at a timing based on the rate pulse.

That is, the transmission delay unit changes the delay time to be given to each rate pulse to adjust the transmission direction of the ultrasonic wave transmitted from a piezoelectric transducer element surface as desired. In addition, the transmission delay unit changes the delay time to be given to each rate pulse to control the position of a focusing point (a transmission focus) in the depth direction of ultrasonic transmission.

Note that the transmitter circuit 19 has a function of being able to instantly change a transmission frequency, a transmission drive voltage, and the like in order to execute a certain scan sequence based on instructions of the processing circuit 150, which will be described later. In particular, the change of the transmission drive voltage is implemented by a linear amplifier type transmitter circuit that can instantly switch the value or by a mechanism that electrically switches a plurality of power supply units.

The receiver circuit 11 includes an amplifier circuit, an analog/digital (A/D) converter, a reception delay circuit, an adder, a quadrature detection circuit, and the like, and performs various types of processing on the reflected wave signal received from the ultrasonic probe 15 to generate a reception signal (reflected wave data). The amplifier circuit amplifies the reflected wave signal for each channel to perform gain correction processing. The A/D converter performs A/D conversion on the gain-corrected reflected wave signal. The reception delay circuit gives digital data a reception delay time necessary to determine reception directivity. The adder performs adding processing on the reflected wave signal to which the reception delay time has been given by the reception delay circuit. The adding processing by the adder emphasizes a reflection component from a direction corresponding to the reception directivity of the reflected wave signal. The quadrature detection circuit converts the output signal of the adder into an in-phase (I) signal and a quadrature-phase (Q) signal in a baseband. The quadrature detection circuit transmits the I signal and the Q signal (hereinafter referred to as IQ signal) to the processing circuit 150 as the reception signal (reflected wave data). Note that the quadrature detection circuit may convert the output signal of the adder into a radio frequency (RF) signal and transmit it to the processing circuit 150. The IQ signal and the RF signal are reception signals having phase information.

When a two-dimensional region inside the subject P is scanned, the transmitter circuit 19 causes the ultrasonic probe 15 to transmit an ultrasonic beam for scanning the two-dimensional region. The receiver circuit 11 then generates a two-dimensional reception signal from a two-dimensional reflected wave signal received from the ultrasonic probe 15. When a three-dimensional region inside the subject P is scanned, the transmitter circuit 19 causes the ultrasonic probe 15 to transmit an ultrasonic beam for scanning the three-dimensional region. The receiver circuit 11 then generates a three-dimensional reception signal from a three-dimensional reflected wave signal received from the ultrasonic probe 15. The receiver circuit 11 generates the reception signal based on the reflected wave signal, and transmits the generated reception signal to the processing circuit 150.

The transmitter circuit 19 causes the ultrasonic probe 15 to transmit the ultrasonic beam from a certain transmission position (transmission scan line). The receiver circuit 11 receives a signal due to a reflected wave of the ultrasonic beam transmitted by the transmitter circuit 19 at a certain reception position (reception scan line) from the ultrasonic probe 15. When parallel simultaneous reception is not performed, the transmission scan line and the reception scan line are the same scan line. In contrast, when parallel simultaneous reception is performed, if the transmitter circuit 19 causes the ultrasonic probe 15 to transmit one-time ultrasonic beam on one transmission scan line, the receiver circuit 11 simultaneously receives signals due to a reflected wave derived from the ultrasonic beam that has been transmitted from the ultrasonic probe 15 by the transmitter circuit 19, as a plurality of reception beams at a plurality of certain reception positions (reception scan lines) through the ultrasonic probe 15.

The medical image processing apparatus 100 is connected to the transmitter circuit 19 and the receiver circuit 11, and executes processing on the signal received from the receiver circuit 11 and control of the transmitter circuit 19. The medical image processing apparatus 100 includes the processing circuit 150, a memory 132, an input apparatus 134, and a display 135. The processing circuit 150 includes a B mode processing function 150a, a Doppler processing function 150b, a generation function 150c, a display control function 150d, a reception function 150e, the control function 150f, a reconstruction function 150g, a selection function 150h, a first analysis function 150i, and a second analysis function 150j.

In the embodiment, respective processing functions performed by the B mode processing function 150a, the Doppler processing function 150b, the generation function 150c, the display control function 150d, the reception function 150e, the control function 150f, the reconstruction function 150g, the selection function 150h, the first analysis function 150i, and the second analysis function 150j are stored in the memory 132 in the form of computer programs executable by a computer. The processing circuit 150 is a processor that reads the computer programs from the memory 132 and executes them to implement functions corresponding to the respective computer programs. In other words, the processing circuit 150 having read the respective computer programs will have the respective functions illustrated inside the processing circuit 150 in FIG. 1. Note that in FIG. 1, the functions of the processing circuit 150 are described as being implemented by a single processing circuit, but a plurality of independent processors may be combined to constitute the processing circuit 150, and each processor may execute a computer program to implement a function. In other words, each function described above may be configured as a computer program, and one processing circuit may execute each computer program. A single processing circuit may implement two or more functions out of the functions of the processing circuit 150. As another example, a specific function may be installed in a dedicated, independent computer program execution circuit.

Note that in FIG. 1, the processing circuit 150, the B mode processing function 150a, the Doppler processing function 150b, the generation function 150c, the display control function 150d, the reception function 150e, the control function 150f, the reconstruction function 150g, the selection function 150h, the first analysis function 150i, and the second analysis function 150j are examples of a B mode processing unit, a Doppler processing unit, a generation unit, a display control unit, a reception unit, a control unit, a reconstruction unit, a selection unit, a first analysis unit, and a second analysis unit, respectively. transmitter circuit 19 and the receiver circuit 11 are examples of a transmitter and receiver.

The The term “processor” used in the above description means, for example, a circuit such as a central processing unit (CPU), a graphical processing unit (GPU), an application specific integrated circuit (ASIC), or a programmable logic device (for example, a simple programmable logic device (SPLD), a complex programmable logic device (CPLD), or a field programmable gate array (FPGA)). The processor reads and executes the computer program stored in the memory 132 to implement the functions.

Instead of storing the computer program in the memory 132, the computer program may be directly embedded in a circuit of the processor. In this case, the processor reads and executes the computer program embedded in the circuit to implement the functions. The transmitter circuit 19, the receiver circuit 11, and the like integrated in the ultrasonic diagnostic apparatus 10 may be configured by hardware such as an integrated circuit, but may be a modularized computer program as software.

The processing circuit 150 is a processing unit performing various types of signal processing on the reception signal received from the receiver circuit 11. The processing circuit 150 includes the B mode processing function 150a, the Doppler processing function 150b, the generation function 150c, the display control function 150d, the reception function 150e, the control function 150f, the reconstruction function 150g, the selection function 150h, the first analysis function 150i, and the second analysis function 150j.

The processing circuit 150, by the B mode processing function 150a, receives data from the receiver circuit 11 to perform logarithmic amplification processing, envelope curve detection processing, logarithmic compression processing, or the like to generate data in which signal intensity is represented in the intensity of brightness (B mode data).

The processing circuit 150, by the Doppler processing function 150b, performs frequency analysis on velocity information from the reception signal (reflected wave data) received from the receiver circuit 11 to generate data in which moving body information such as velocity, dispersion, power, and the like by the Doppler effect is extracted for multiple points (Doppler data).

Note that the B mode processing function 150a and the Doppler processing function 150b exemplified in FIG. 1 can process both two-dimensional reflected wave data and three-dimensional reflected wave data.

The processing circuit 150, by the generation function 150c, generates ultrasonic image data from the data generated by the B mode processing function 150a and the Doppler processing function 150b. The processing circuit 150, by the generation function 150c, generates two-dimensional B mode image date in which the intensity of the reflected wave is represented in brightness from two-dimensional B mode data generated by the B mode processing function 150a. The processing circuit 150, by the generation function 150c, generates two-dimensional Doppler image data representing the moving body information from two-dimensional Doppler data generated by the Doppler processing function 150b. The two-dimensional Doppler image data is velocity image data, dispersion image data, power image data, or image data obtained by combining them.

The processing circuit 150, by the generation function 150c, converts (scan converts) a scan line signal sequence of ultrasonic scan into a scan line signal sequence of video format represented by television or the like to generate ultrasonic image data for display. The processing circuit 150, by the generation function 150c, performs various types of image processing, besides the scan convert, for example, image processing to regenerate a brightness average value image (smoothing processing), image processing using a differential filter in an image (edge enhancement processing), or the like using a plurality of image frames after the scan convert. The processing circuit 150, by the generation function 150c, performs various types of rendering processing on volume data in order to generate two-dimensional image data for displaying the volume data on the display 135.

The processing circuit 150, by the display control function 150d, performs control to display the ultrasonic image data for display stored in the memory 132 on the display 135.

The processing circuit 150, by the reception function 150e, receives various operations from a user through the input apparatus 134.

The processing circuit 150, by the control function 150f, controls the entire processing of the ultrasonic diagnostic apparatus. Specifically, the processing circuit 150, by the control function 150f, controls the processing of the transmitter circuit 19, the receiver circuit 11, and the processing circuit 150 based on various setting requests input from an operator via the input apparatus 134 and various control programs and various data read from the memory 132.

The processing circuit 150 also includes the reconstruction function 150g, the selection function 150h, the first analysis function 150i, and the second analysis function 150j. These functions will be described later.

The memory 132 includes a semiconductor memory element such as a random access memory (RAM) and a flash memory, a hard disk, an optical disc, or the like. The memory 132 is a memory storing therein data such as image data for display generated by the processing circuit 150. The memory 132 can also store therein the data generated by the B mode processing function 150a or the Doppler processing function 150b. The B mode data or the Doppler data stored in the memory 132 can be, for example, called by the operator after diagnosis and becomes the ultrasonic image data for display through the processing circuit 150. The memory 132 can also store therein the reception signal (reflected wave data) output by the receiver circuit 11.

In addition, the memory 132 stores therein control programs for performing ultrasonic transmission and reception, image processing, and display processing, diagnostic information (for example, patient IDs, opinions by doctors, and the like), and various data such as diagnostic protocols and various body marks as needed.

The input apparatus 134 receives various instructions and information input from the operator. The input apparatus 134 is, for example, a pointing device such as a mouse or a trackball, a selection device such as a mode switching switch, or an input device such as a keyboard.

The display 135 displays a graphical user interface (GUI) for receiving input of imaging conditions, images or the like generated by the generation function 150c or the like, and other items under the control of the control function 150f or the like. The display 135 is, for example, a display device such as a liquid crystal display. The display 135 is an example of a display unit. The display 135 includes a mouse, a keyboard, a button, a panel switch, a touch command screen, a foot switch, a trackball, a joystick, and the like.

Next, a background according to the embodiment will be described.

In the field of seismic wave measurement, seismic wave interferometry (also referred to as shot-profile migration (SPM) or wave-field correlation) or RTM (Reverse-Time Migration) is known. Seismic wave interferometry is a method for reconstructing a hypocentral position and reflection surfaces in the ground, and is a method for estimating the hypocentral position and the like by performing correlation processing between a transmission wave field obtained from forward propagation simulation and a reception wave field obtained from back propagation simulation.

Since a seismic wave and an ultrasonic wave are common in terms of being an elastic wave, as described in, for example, Non-Patent Literature 1 (“Distributed Aberration Correction Techniques Based on Tomographic Sound Speed Estimates”, R. Ali etc., IEEE T-UFFC, Vol. 69, No. 5, p 1714, May 2022”), the method of seismic wave interferometry may be used for image reconstruction in the ultrasonic diagnostic apparatus to reconstruct an ultrasonic image.

When the depth of a point to be imaged is shallow, and it is a point near the body surface, the amplitude of an ultrasonic wave itself has sufficient intensity. Thus, for example, to prevent overlooking of a tumor or the like on the body surface, it is desirable to perform image reconstruction using only a component in a direction directed to the sensor out of a reflected wavefront from the tumor and to suppress components in the other directions because they contain many unnecessary wave signals from outside the tumor. In contrast, when the depth of the point to be imaged is deep, and it is a point far from the body surface, the amplitude of the ultrasonic wave becomes smaller due to signal attenuation. Thus, to ensure an S/N ratio, image reconstruction is desirably performed using all the signals received by the sensor even if the signals from outside the point to be imaged are included.

However, since conventional seismic wave interferometry performs image reconstruction using signals in various directions received by the sensor with equivalent weights, it may be difficult to ensure the S/N ratio while suppressing unnecessary wave components.

The ultrasonic diagnostic apparatus of the embodiment is based on such a background, and the ultrasonic diagnostic apparatus according to the embodiment includes the transmitter circuit 19 and the receiver circuit 11 as the transmitter and receiver, and the processing circuit 150. The transmitter circuit 19 and the receiver circuit 11 as the transmitter and receiver transmits an ultrasonic wave into a subject, based on a transmission condition, and receive an echo from inside the subject. The processing circuit 150, by the first analysis function 150i, causes the ultrasonic wave transmitted based on the transmission condition to propagate forward to obtain a transmission wave field. The processing circuit 150, by the second analysis function 150j, causes a signal based on the echo to propagate backward to obtain a reception wave field by applying a weight function depending on a wavefront incident angle. The processing circuit 150, by the reconstruction function 150g, performs correlation analysis between the transmission wave field and the reception wavefield to generate an echo component.

The method of ultrasonic diagnosis according to the embodiment causes a transmission signal to propagate forward to obtain a transmission wave field, causes a reception signal to perform a backward propagation simulation to obtain a reception wave field by applying a weight function depending on a wavefront incident angle representing an incident angle of a wavefront when propagating backward the reception signal, and performs correlation analysis between the transmission wave field and the reception wave field to generate a signal.

The computer program according to the embodiment causes a computer to execute processing of causing a transmission signal to perform a backward propagation simulation to obtain a transmission wave field, causing a reception signal to propagate backward to obtain a reception wave field by applying a weight function depending on a wavefront incident angle representing an incident angle of a wavefront when propagating backward the reception signal the reception signal, and performing correlation analysis between the transmission wave field and the reception wave field to generate a signal.

Thus, by changing performing reconstruction by seismic wave interferometry using the reception signal corresponding to how large incident angle for each depth, an image with high reliability can be obtained at a place where the depth is shallow by performing reconstruction using the reception signal with high reliability, and the S/N ratio can be improved at a place where the depth is deep by performing reconstruction using all the reception signals.

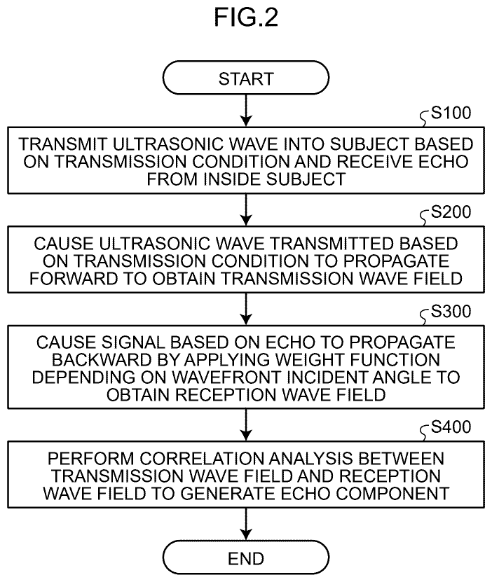

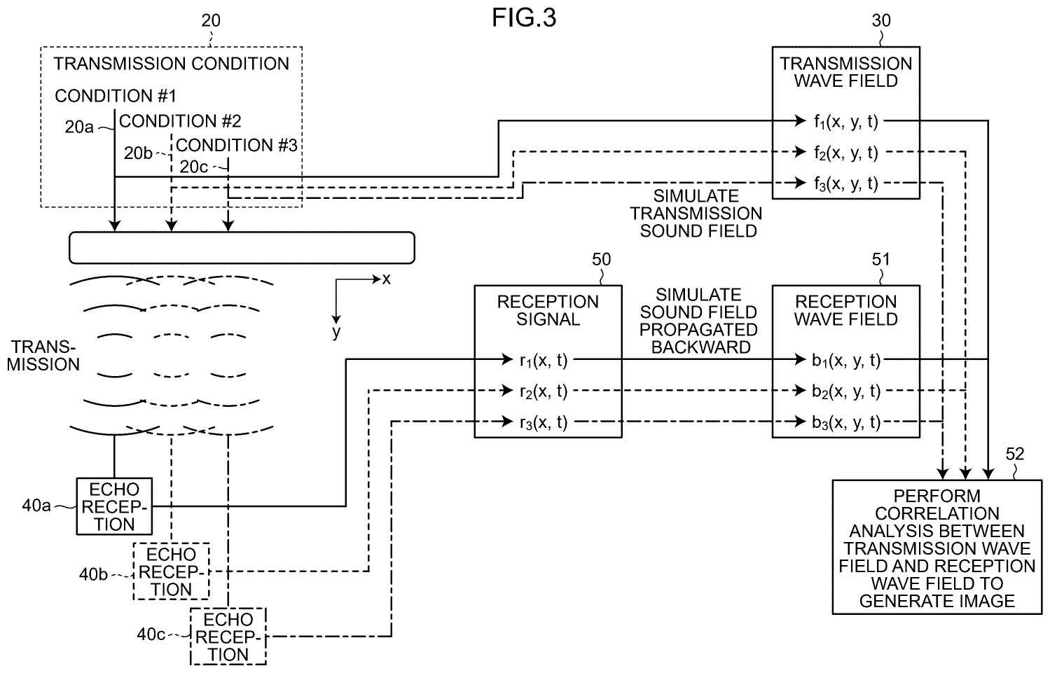

First, processing performed by the ultrasonic diagnostic apparatus 10 according to the first embodiment will be described using FIG. 2 and FIG. 3. FIG. 2 is a flowchart illustrating the flow of the processing performed by the ultrasonic diagnostic apparatus 10 according to the embodiment. FIG. 3 is a diagram illustrating the processing of image reconstruction using seismic wave interferometry, performed by the ultrasonic diagnostic apparatus 10 according to the embodiment.

First, at Step S100, the transmitter circuit 19 and the receiver circuit 11 as the transmitter and receiver transmit an ultrasonic wave into a subject, based on a transmission condition, and receive an echo from inside the subject. In the ultrasonic diagnostic apparatus 10, typically, the transmitter circuit 19 transmits a plurality of ultrasonic waves to different directions, and the receiver circuit 11 receives the echo from the subject for each of the ultrasonic waves. For example, as schematically illustrated in FIG. 3, the transmitter circuit 19 transmits a plurality of ultrasonic waves 20a, 20b, and 20c. The receiver circuit 11 receives the echo from the subject for each of the ultrasonic waves 20a, 20b, and 20c (echo reception 40a, 40b, and 40c) to acquire each of reception signals r₁(x,t), r₂(x,t), and r₃(x,t) as a reception signal 50 where x is a position coordinate in the travel direction of an ultrasonic beam and t is time.

The transmission condition typically refers to a transmission condition such as the amplitude, phase, frequency, transmission aperture, or transmission apodization of the ultrasonic wave to be transmitted.

Next, at Step S200, the processing circuit 150, by the first analysis function 150i, causes the ultrasonic wave transmitted based on the transmission condition at Step S100 to propagate forward, and calculates a transmission wave field 30 by forward propagation simulation. As an example, as illustrated in FIG. 3, the processing circuit 150, by the first analysis function 150i, causes the ultrasonic wave 20a, the ultrasonic wave 20b, and the ultrasonic wave 20c transmitted under a transmission condition #1, a transmission condition #2, and a transmission condition #3, respectively to propagate forward, and calculates transmission wave fields f₁(x,y,t), f₂(x,y,t), and f₃(x,y,t) by forward propagation simulation, where x represents a position coordinate in a direction perpendicular to the travel direction of the ultrasonic beam, y represents a position coordinate in the travel direction of the ultrasonic beam, and t represents time. The processing circuit 150, by the first analysis function 150i, for example, simulates the temporal evolution of a wave equation to obtain the transmission wave field 30 by numerical analysis based on the wave equation. As an example, the processing circuit 150, with the first analysis function 150i, calculates the transmission wave field 30, based on the transmission condition at Step S100, by using, for example, the finite difference time domain (FDTD) method. As another example, the processing circuit 150, by the first analysis function 150i, calculates the transmission wave field 30, based on the transmission condition at Step S100, by using, for example, a simulation method using an angular spectrum method. However, FIG. 3 illustrates a focused sound field as the transmission wave field, but the present embodiment is not limited to this example, and, for example, a plane wave sound field or a diffuse sound field may be used as a transmission sound field.

Next, at Step S300, the processing circuit 150, by the second analysis function 150j, causes a signal based on the echo to propagate backward to obtain a reception wave field 51 by applying a weight function depending on a wavefront incident angle. The details of the wavefront incident angle and the weight function depending on the wavefront incident angle will be described in detail after describing the entire flowchart in FIG. 2. As illustrated in FIG. 3, the processing circuit 150 causes the reception signal 50 based on the echo acquired at Step S100 to propagate backward to calculate the reception wave field 51. As an example, the processing circuit 150, by the second analysis function 150j, obtains the reception wave field 51 by numerical analysis based on a wave equation. For example, the processing circuit 150, by the second analysis function 150j, simulates the temporal evolution of the wave equation using the FDTD method to calculate reception wave fields b₁(x,y,t), b₂(x,y,t), and b₃(x,y,t) from reception signals r₁(x), r₂(x), and r₃(x), respectively.

Next, at Step S400, the processing circuit 150, by the reconstruction function 150g, performs correlation analysis between the transmission wave field 30 and the reception wave field 51 to generate an echo component and to generate an image 52. Specifically, an image I is generated by Expression (1) below:

$$I ( x , y ) = {\sum }_{n = 1}^{N} \int {f}_{n}^{*} ( x , y , t ) {b}_{n} ( x , y , t ) dt \qquad ( 1 )$$

where n is an index distinguishing ultrasonic waves 20a, 20b, 20c, and the like, and N represents the total number of pieces of ultrasonic transmission contained in a series of pieces of ultrasonic transmission. In addition, represents complex conjugation.

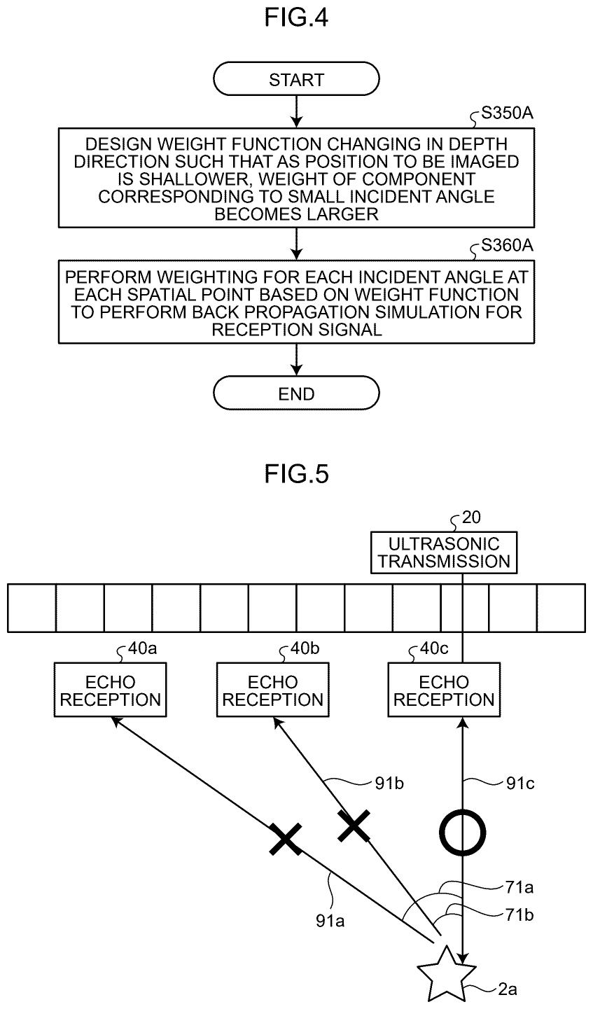

Next, the details of the processing at Step S300 will be described using FIG. 4 to FIG. 7. FIG. 4 is a flowchart illustrating an example of the processing at Step S300 in more detail. In the processing at Step S300, first, at Step S350A, the processing circuit 150, by the second analysis function 150j, designs and applies a weight function changing in accordance with a position in a depth direction. As an example, the processing circuit 150, by the second analysis function 150j, applies a weight function changing in the depth direction such that as a position to be imaged is shallower, the weight of a component corresponding to a small incident angle becomes larger. That is, the processing circuit 150, by the second analysis function 150j, applies a weight function in which the contribution of the weight of a component the wavefront incident angle of which is small is relatively larger as the depth is shallower. Next, at Step S360A, the processing circuit 150, by the second analysis function 150j, performs weighting for each incident angle at each spatial point based on the weight function to perform back propagation simulation for the reception signal.

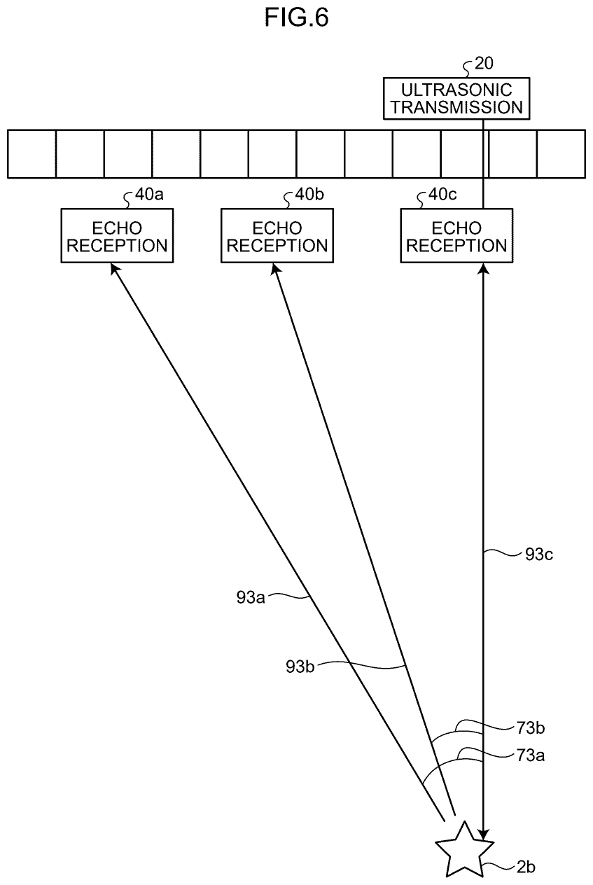

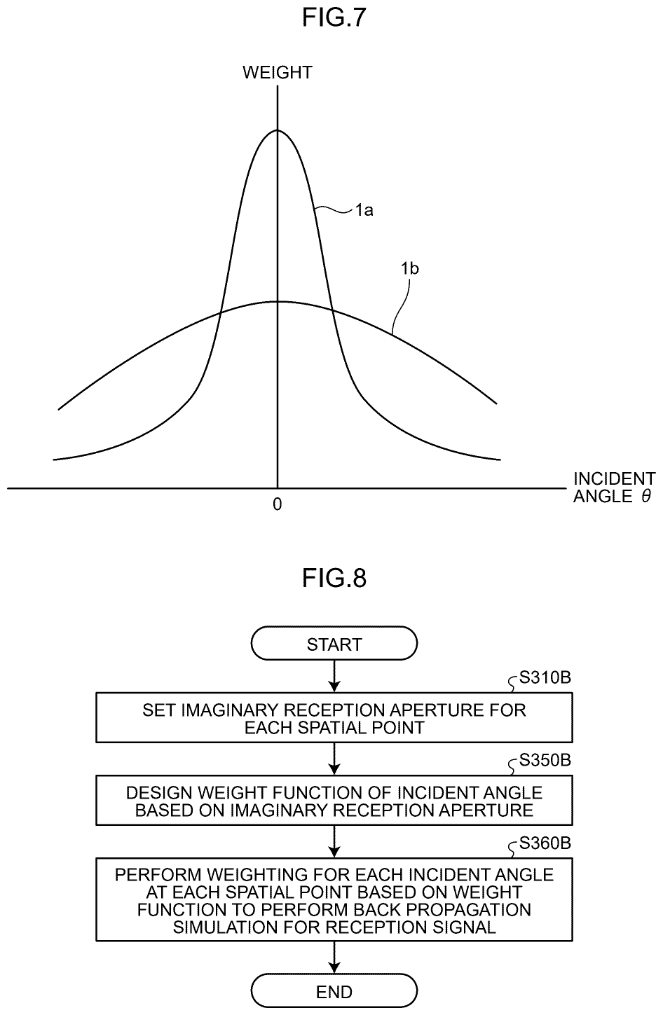

These points will be described using FIG. 5 to FIG. 7. FIG. 5 is a diagram illustrating the design of the weight function when the position of a point 2a for which an image is obtained is shallow, FIG. 6 is a diagram illustrating the design of the weight function when the position of a point 2b for which an image is obtained is deep, and FIG. 7 is a diagram of an example of the shape of the weight function.

A case when the position of the point 2a for which an image is obtained is shallow is considered with reference to FIG. 5. The transmission wave transmitted by ultrasonic transmission 20 is reflected on the point 2a, and the receiver circuit 11 performs the echo reception 40a, 40b, and 40c at different positions. Respective straight lines 91a, 91b, and 91c are straight lines, when the transmission ultrasonic wave transmitted by the ultrasonic transmission 20 is reflected at the position of the point 2a and echoes are received at positions corresponding to the echo reception 40a, 40b, and 40c, each of the straight lines representing the travel direction of the wavefront of the ultrasonic wave. The straight lines 91a, 91b, and 91c are also straight lines indicating directions representing the incident directions of wavefronts obtained by performing back propagation simulation on the signals received at the positions corresponding to the echo reception 40a, 40b, and 40c. The incident direction of the wavefront when back propagation simulation is performed is a direction opposite to the travel direction of the reflected transmission ultrasonic wave.

A wavefront incident angle 71a is an incident angle representing the incident direction of a wavefront when back propagation simulation is performed on the signal based on the echo corresponding to the echo reception 40a, which is a position far from a front position, at which the ultrasonic transmission has been performed. The wavefront incident angle 71a is defined as an angle formed by the straight line 91a and the transmission direction of the transmission ultrasonic wave transmitted by the ultrasonic transmission 20. The wavefront incident angle 71a corresponding to the echo reception 40a, which is the position far from the front position, at which the ultrasonic transmission has been performed, is a value larger than a wavefront incident angle 71b corresponding to the echo reception 40b, which is a position near the front position, at which the ultrasonic transmission has been performed.

The wavefront incident angle 71b is an incident angle representing the incident direction of a wavefront when back propagation simulation is performed on a signal based on the echo corresponding to the echo reception 40b, which is the position near the front position, at which the ultrasonic transmission has been performed. The wavefront incident angle 71b is defined as an angle formed by the straight line 91b and the transmission direction of the transmission ultrasonic wave transmitted by the ultrasonic transmission 20. The wavefront incident angle 71b corresponding to the echo reception 40b, which is the position near the front position, at which the ultrasonic transmission has been performed, is a value smaller than the wavefront incident angle 71a corresponding to the echo reception 40a, which is the position far from the front position, at which the ultrasonic transmission has been performed.

For a signal based on the echo corresponding to the echo reception 40c, which is the front position, at which the ultrasonic transmission has been performed, its incident direction of a wavefront propagated backward is a direction nearly 180 degrees opposite to the transmission direction of the transmission ultrasonic wave transmitted by the ultrasonic transmission 20, and thus in this case, the wavefront incident angle is nearly zero.

In FIG. 6, a case when the position of the point 2b for which an image is obtained is deep is considered. The ultrasonic wave transmitted by the ultrasonic transmission 20 is reflected on the point 2b, and the receiver circuit 11 performs the echo reception 40a, 40b, and 40c at different positions. Straight lines 93a, 93b, and 93c are straight lines, when the transmission ultrasonic wave transmitted by the ultrasonic transmission 20 is reflected at the position of the point 2b, and echoes are received at positions corresponding to the echo reception 40a, 40b, and 40c, each of the straight lines representing the travel direction of the wavefront of the ultrasonic wave. The straight lines 93a, 93b, and 93c are also straight lines indicating directions representing the incident directions of wavefronts obtained by performing back propagation simulation on the signals received at the positions corresponding to the echo reception 40a, 40b, and 40c. The incident direction of the wavefront when back propagation simulation is performed is a direction opposite to the travel direction of the reflected transmission ultrasonic wave.

A wavefront incident angle 73a is an incident angle representing the incident direction of a wavefront when back propagation simulation is performed on the signal based on the echo corresponding to the echo reception 40a, which is a position far from a front position, at which the ultrasonic transmission has been performed. The wavefront incident angle 73a is defined as an angle formed by the straight line 93a and the transmission direction of the transmission ultrasonic wave transmitted by the ultrasonic transmission 20. The wavefront incident angle 73a corresponding to the echo reception 40a, which is the position far from the front position, at which the ultrasonic transmission has been performed, is a value larger than a wavefront incident angle 73b corresponding to the echo reception 40b, which is a position near the front position, at which the ultrasonic transmission has been performed.

The wavefront incident angle 73b is an incident angle representing the incident direction of a wavefront when back propagation simulation is performed on a signal based on the echo corresponding to the echo reception 40b, which is the position near the front position, at which the ultrasonic transmission has been performed. The wavefront incident angle 73b is defined as an angle formed by the straight line 93b and the transmission direction of the transmission ultrasonic wave transmitted by the ultrasonic transmission 20. The wavefront incident angle 73b corresponding to the echo reception 40b, which is the position near the front position, at which the ultrasonic transmission has been performed, is a value smaller than the wavefront incident angle 73a corresponding to the echo reception 40a, which is the position far from the front position, at which the ultrasonic transmission has been performed.

For a signal based on the echo corresponding to the echo reception 40c, which is the front position, at which the ultrasonic transmission has been performed, its incident direction of a wavefront propagated backward is a direction nearly 180 degrees opposite to the transmission direction of the transmission ultrasonic wave transmitted by the ultrasonic transmission 20, and thus in this case, the wavefront incident angle is nearly zero.

At Step S300 for calculating the reception wave field 51 using seismic wave interferometry, with the wavefront incident angle in what range performing image reconstruction using the signal based on the echo is an important issue. That is, when the point 2a to be imaged is near the sensor and shallow as in FIG. 5, since the signal intensity is sufficient at the shallow position, image quality is sufficient even when, for example, image reconstruction is performed using only the signal from the echo reception 40c, which is at the front position. In contrast, when image reconstruction is performed using the signal from the echo reception 40a or 40b, which is far from the front position, noise or the like is imaged, and the image quality of a reconstructed image may be rather degraded.

Thus, for example, when the position of the point 2a to be imaged is shallow as in FIG. 5, a function similar to, for example, a weight function 1a in FIG. 7, in which the weight is a large value only when a wavefront incident angle θ is near zero and the value of the weight function rapidly decreases as the wavefront incidence angle θ increases is selected.

In contrast, when the position of the point 2b to be imaged is near the sensor and deep as in FIG. 6, since signals are attenuated at the deep position, it is desirable to perform image reconstruction with all the signals of the echo reception 40a, 40b, and 40c picked up. Thus, for example, when the position of the point 2b to be imaged is deep as in FIG. 6, a function similar to, for example, a weight function 1b in FIG. 7, in which the value of the weight function is relatively large also in an area in which the wavefront incident angle θ is large is selected.

To summarize the above, as the processing at Step S300, first, at Step S350, the processing circuit 150, by the second analysis function 150j, designs and applies a weight function changing in accordance with the position in the depth direction. As an example, the processing circuit 150, by the second analysis function 150j, applies a weight function in which the contribution of the weight of a component the wavefront incident angle of which is small becomes relatively larger as the depth becomes shallower. Next, at Step S360A, the processing circuit 150, by the second analysis function 150j, performs weighting for each incident angle at each spatial point based on the weight function to perform back propagation simulation for the reception signal. In other words, the processing circuit 150, by the second analysis function 150j, causes the signal based on the echo to propagate backward to obtain the reception wave field, based on the weight function depending on a positional relation between a point for which an image is obtained and a point at which the echo has been received.

As described above, in the first embodiment, in the ultrasonic reconstruction using seismic wave interferometry, the signal based on the echo propagates backward to obtain the reception wave field by applying the weight function depending on the wavefront incident angle. Thus, for example, at a shallow position, image quality can be improved by suppressing unnecessary signal components, whereas at a deep position, the S/N ratio can be ensured, and an image with high image quality can be obtained both at the shallow position and the deep position.

In The embodiment is not limited to the above example, the embodiment described above, a continuous function is employed as the weight function employed at Step S300, but the embodiment is not limited to this example. For example, as the weight function, a discontinuous weight function, for example, a weight function in which the weight of a specific reception signal is 1 and the weight of the other reception signals is 0 may be selected.

As an example, at Step S300, the processing circuit 150, by the selection function 150h, may select a signal based on the echo to be propagated backward in the process of generating the reception wave field, and, by the second analysis function 150j, may cause only the selected signal based on the echo to propagate backward to generate the reception wave field.

The above embodiment describes a case in which ultrasonic reconstruction is performed using seismic wave interferometry, but the embodiment can be extended to model-based reconstruction in general that uses computer simulations of wave propagation. Examples of the model-based reconstruction method in the embodiment include, for example, a method of causing a transmission signal to propagate forward to obtain a transmission wave field, causing a reception signal to propagate backward to obtain a reception wave field by applying a weight function to a wavefront incident angle, and performing correlation analysis between the transmission wave field and the reception wave field to generate a signal.

### Second Embodiment

In a second embodiment, a case in which the weight function is determined based on an imaginary reception aperture determined for each position in the depth direction will be described. Note that the second embodiment is the same as the first embodiment in the processing other than Step S300 in FIG. 2, and thus descriptions of the processing other than Step S300 are omitted. In the second embodiment, the processing circuit 150 performs the processing illustrated in FIG. 8 at Step S300. FIG. 8 is a flowchart illustrating the flow of the processing performed at Step S300 in FIG. 2 in the second embodiment.

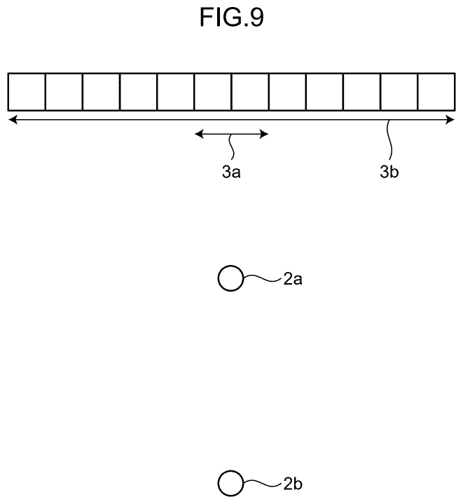

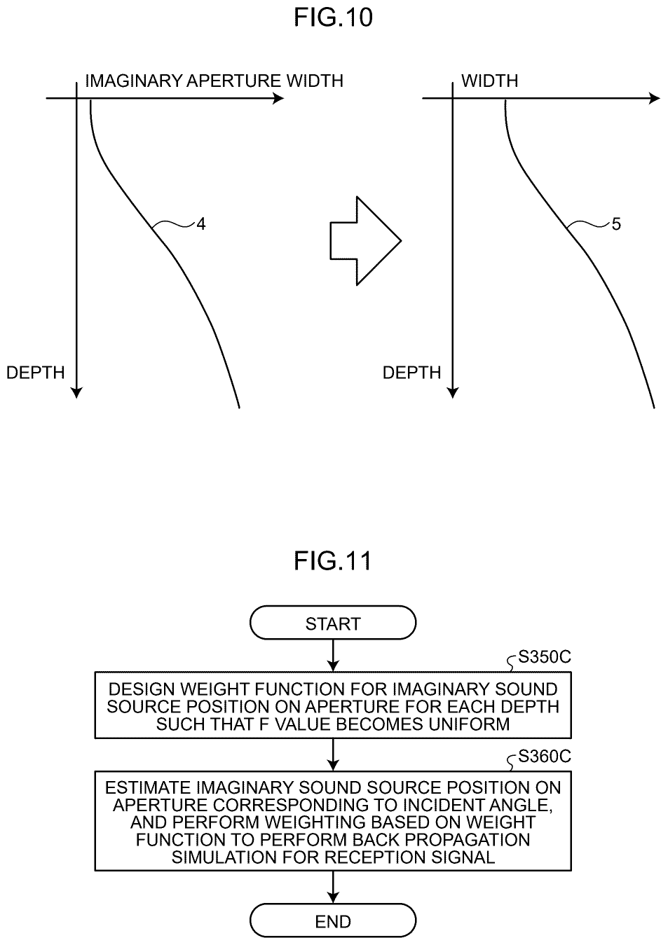

First, at Step S310B, the processing circuit 150, by the second analysis function 150j, sets an imaginary reception aperture at each spatial point. To describe the imaginary reception aperture, since ultrasonic transducer elements have directivity in the ultrasonic diagnostic apparatus, only sounds in a limited range can be picked up. Thus, for example, for a reflected wave at the point 2a, which is at a shallow place in FIG. 9, signals can be received only by a group of transducer elements present in a range of a width 3a. For a reflected wave at the point 2b, which is at a deep place, on the contrary, signals can be received by a group of transducer elements in a range of a width 3b. Thus, for a point to be imaged, an effective aperture width of the group of transducer elements can be considered, which is called the imaginary reception aperture. That is, the width 3a is the imaginary reception aperture for the point 2a, and the width 3b is the imaginary reception aperture for the point 2b. When the imaginary reception aperture width is plotted, for example, as a function of depth, it is shaped like, for example, a curve 4 illustrated in FIG. 10. At Step S310B, the processing circuit 150, by the second analysis function 150j, sets the imaginary reception aperture at each spatial point by acquiring, from the memory 132, data indicating the shape of the curve 4 obtained by, for example, performing measurement on a known group of transducer elements.

To describe the meaning of the imaginary reception aperture, in seismic wave interferometry, all the ultrasonic transducer elements actually receive signals. However, when image reconstruction in the vicinity of reception aperture is performed using signals from the ultrasonic transducer elements in a part away from the center, image degradation occurs, and thus the signals from the ultrasonic transducer elements in the part away from the center can be prevented from propagating backward not to be subjected to image reconstruction or lessen the contribution from back propagation signals from ultrasonic transducer elements far from the center. That is, in this case, signals at positions that are far from the front position than the extent of the imaginary reception aperture are not reconstructed or contribute less to the reconstruction. In other words, the imaginary reception aperture is considered to nearly represent a cutoff position of the weight function.

Next, at Step S350B, the processing circuit 150, by the second analysis function 150j, designs the weight function of the wavefront incident angle based on the imaginary reception aperture set at Step S350. As an example, the processing circuit 150, by the second analysis function 150j, sets the weight function by designing a half-width 5 of the weight function represented as a function of the wavefront incident angle at each depth such that the shape of the half-width 5 matches an imaginary aperture width at each depth. Next, at Step S360B, the processing circuit 150, by the second analysis function 150j, performs weighting for each incident angle at each spatial point based on the weight function to perform back propagation simulation for the reception signal.

The embodiment is not limited to this example, and the processing circuit 150 may design the weight function based on an F value. As an example, as illustrated in FIG. 11, at Step S350C and S360C, the processing circuit 150, by the second analysis function 150j, estimates an imaginary sound source position on the aperture for each depth such that the F value becomes uniform, and performs weighting based on the weight function to perform back propagation simulation for the reception signal.

Thus, for example, at a shallow position, image quality can be improved by suppressing unnecessary signal components, whereas at a deep position, the S/N ratio can be ensured, and an image with high image quality can be obtained both at the shallow position and the deep position.

At least one of the embodiments described above can improve image quality.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
a transmitter circuit and receiver circuit configured to transmit an ultrasonic wave into a subject, based on a transmission condition, and receive an echo from inside the subject; and
a processing circuit configured to
cause the ultrasonic wave transmitted based on the transmission condition to propagate forward to obtain a transmission wave field,
cause a signal based on the echo to propagate backward to obtain a reception wave field by applying a weight function depending on a wavefront incident angle, and
perform correlation analysis between the transmission wave field and the reception wave field to generate an echo component.

2. The ultrasonic diagnostic apparatus according to claim 1, wherein the weight function is a function changing in accordance with a position in a depth direction.

3. The ultrasonic diagnostic apparatus according to claim 2, wherein the weight function is a function in which contribution of weight of a component the wavefront incident angle of which is small becomes relatively larger as depth becomes shallower.

4. The ultrasonic diagnostic apparatus according to claim 3, wherein the weight function is determined based on an imaginary reception aperture determined for each position in the depth direction.

5. The ultrasonic diagnostic apparatus according to claim 1, wherein
the processing circuit selects the signal based on the echo to be propagated backward in a process of generating the reception wave field, and
the processing circuit causes only the selected signal based on the echo to propagate backward to generate the reception wave field.

6. The ultrasonic diagnostic apparatus according to claim 1, wherein the processing circuit
obtains the transmission wave field by numerical analysis based on a wave equation, or
obtains the reception wave field by numerical analysis based on a wave equation.

7. The ultrasonic diagnostic apparatus according to claim 1, wherein the wavefront incident angle represents an incident direction of a wavefront when back propagation simulation is performed on the signal based on the echo.

8. The ultrasonic diagnostic apparatus according to claim 1, wherein the processing circuit causes the signal based on the echo to propagate backward to obtain the reception wave field, based on the weight function depending on a positional relation between a point for which an image is obtained and a point at which the echo has been received.

9. A method of signal processing, the method comprising:
causing a transmission signal to propagate forward to obtain a transmission wave field;
causing a reception signal to propagate backward to obtain a reception wave field by applying a weight function depending on a wavefront incident angle representing an incident angle of a wavefront when back propagation simulation is performed on the reception signal; and
performing correlation analysis between the transmission wave field and the reception wave field to generate a signal.

10. A non-transitory computer readable medium including programmed instructions, wherein the instructions, when executed by a computer, cause the computer to perform:
causing a transmission signal to propagate forward to obtain a transmission wave field;
causing a reception signal to propagate backward to obtain a reception wave field by applying a weight function depending on a wavefront incident angle representing an incident angle of a wavefront when back propagation simulation is performed on the reception signal; and
performing correlation analysis between the transmission wave field and the reception wave field to generate a signal.
